(19) United States
(12) Patent Application Publication
(10) Pub. No.: US 2008/0182893 A1
(43) Pub. Date: Jul. 31, 2008

(54) Anti-Atherogenic Composition

(75) Inventors: Yoshiki Nishizawa, Osaka (JP); Hidenori Koyama, Osaka (JP); Koka Motoyama, Osaka (JP); Masamitsu Moriwaki, Osaka (JP); Kazuhiro Emura, Osaka (JP); Mikio Nakamura, Osaka (JP)

Correspondence Address: FITCH, EVEN, TABIN & FLANNERY, P. O. BOX 18415, WASHINGTON, DC, 20036, US

(73) Assignee: SAN-EI GEN F.F.I., INC., Toyonaka-shi, Osaka (JP)

(21) Appl. No.: 11/817,896

(22) PCT Filed: Mar. 6, 2006
(86) PCT No.: PCT/JP2006/304247

§ 371 Date: Sep. 6, 2007

(30) Foreign Application Priority Data
Mar. 9, 2005 (JP) 2005-064903

(51) Int. Cl. A61K 31/352; A61P 9/00

(52) U.S. Cl. 514/456

(57) ABSTRACT

Disclosed is an anti-atherogenic composition or an atherosclerosis progression inhibitory composition which can be used effectively for preventing or ameliorating atherosclerosis. The anti-atherogenic composition or atherosclerosis progression inhibitory composition comprises an enzyme-treated isoquercitrin as the active ingredient.

### TECHNICAL FIELD

The present invention relates to an anti-atherogenic composition that is effectively used for the prevention or amelioration of atherosclerosis.

### BACKGROUND ART

Blood lipids and particularly cholesterol and triglycerides are known to be intimately related to coronary artery and cardiovascular diseases, such as atherosclerosis, hyperlipidemia, fatty liver, and so forth. For example, it has been reported that when the blood cholesterol concentration is high, macrophages and foam cells accumulate along with lipids in the blood vessel wall, forming plaques and leading to atherosclerosis (Non-Patent Document 1). Dietary therapy, in which the intake of cholesterol and lipids is reduced, is one method for lowering blood cholesterol. Another method is to interfere with cholesterol absorption by inhibiting the enzymes related to cholesterol absorption.

It is known that acyl CoA-cholesterol-O-acyltransferase (referred to below as ACAT) is an enzyme that promotes the esterification of blood cholesterol and that the formation of foam cells on artery walls is promoted by the action of this enzyme. Due to this, ACAT inhibitors have the potential to act as agents that prevent atherosclerosis.

It is also known that hypercholesterolemia can be effectively treated by inhibiting the activity of the cholesterol ester transfer protein or by moderating the rate of cholesterol biosynthesis by inhibiting the activity of 3-hydroxy-3-methylglutaryl-CoA reductase (referred to below as HMG-CoA reductase) (Non-Patent Document 2). Lovastatin (trademark, Merck (USA)), simvastatin (trademark, Merck (USA)), and pravastatin (trademark, Sankyo Co., Ltd. (Japan)) have been commercialized as drugs that inhibit the latter HMG-CoA reductase (Non-Patent Document 3).

On the other hand, the capacity to inhibit atherosclerosis has been reported to also be present in polyphenols, such as rutin and quercetin, which are known as natural antioxidants (Non-Patent Document 4, Patent Document 1). However, there have been no reports with regard to enzymatically modified isoquercitrin.
Non-Patent Document 1: Ross R., Nature, 362, 801-809 (1993))
Non-Patent Document 2: Parmley, W. W., Cardiovascular Pharmacology, Kanu Chatterjee Editor, Wolfe Publishing, 8.6-8.7 (1994)
Non-Patent Document 3: C. D. R. Dunn, Stroke: Trends, Treatment and Markets, SCRIPT Report PJB Publications Ltd., 1995
Non-Patent Document 4: Kenshiro Fujimoto, Modern Medicine—“Natural Antioxidant Substances”, Volume 28, Number 8, pages 129-134 (1996)
Patent Document 1: JP 2002-524522 A

### DISCLOSURE OF THE INVENTION

### Problems To Solved By the Invention

An object of the present invention is to provide an anti-atherogenic composition that can inhibit the progression of atherosclerosis in individuals at risk for the progression of atherosclerosis (for example, individuals with hyperlipidemia or trending in that direction). More particularly, a principal object of the present invention is to provide an anti-atherogenic composition that has an atherosclerosis progression inhibiting activity, that exhibits high safety, and that can be taken on a continuous basis.

### Means For Solving the Problems

The present inventors carried out intensive and extensive investigations in order to achieve these objects and as a result discovered that, in animals constructed by genetic manipulation so as to develop hyperlipidemia (ApoE knock out hyperlipidemic mice, referred to below simply as “ApoE-KO mice”), the development of thoracic-abdominal aortic atherosclerotic lesions, which is the initial pathological change in atherosclerosis, can be significantly lowered and the progression of atherosclerosis can be inhibited by the oral administration of enzymatically modified isoquercitrin, and drew the conclusion that enzymatically modified isoquercitrin can be effectively used for the prevention and amelioration of atherosclerosis. The present invention was achieved based on this knowledge.

That is, the invention in this application comprises the following.

Item 1. An anti-atherogenic composition comprising an enzymatically modified isoquercitrin as an effective ingredient.

Item 2. The anti-atherogenic composition according to item 1, which contains the enzymatically modified isoquercitrin in an amount effective for exercising an atherosclerosis progression inhibiting effect.

Item 3. The anti-atherogenic composition according to item 1 or 2, which is in an orally administratable form.

Item 4. The anti-atherogenic composition according to any of items 1 to 3, which contains the enzymatically modified isoquercitrin of 3 mg to 30 g per unit of daily administration.

Item 5. The anti-atherogenic composition according to any of items 1 to 4, which is formulated so as to contain 3 mg to 30 g of the enzymatically modified isoquercitrin per unit of daily administration.

Item 6. The anti-atherogenic composition according to any of items 1 to 5, which is a drug.

Item 7. The anti-atherogenic composition according to any of items 1 to 5, which is a food.

Item 8. The anti-atherogenic composition according to any of items 1 to 7, wherein the anti-atherogenic composition is held in a container and the anti-atherogenic action is indicated as an effect on said container or on packaging thereof.

Item 9. The anti-atherogenic composition according to any of items 1 to 5 and items 7 to 8, which is a Food for Specified Health Use for prevention or amelioration of atherosclerosis.

Item 10. A method of preventing or ameliorating atherosclerosis in a subject at a risk for progression of atherosclerosis, the method comprising:

administering to said subject an enzymatically modified isoquercitrin in an amount effective for exercising an atherosclerosis progression inhibiting effect.

Item 11. A use of an enzymatically modified isoquercitrin for preparation of an anti-atherogenic composition.

As shown in the test examples, the “anti-atherogenic composition” of the present invention acts to inhibit the appearance of aortic atherosclerotic lesions and to inhibit the production of oxidized low-density lipoprotein (oxidized LDL) and as a result exhibits the ability to inhibit the progression of atherosclerosis. Thus, when viewed from different perspectives, the “anti-atherogenic composition” of the present invention can also be considered to be a “composition that inhibits the appearance of aortic atherosclerotic lesions”, a “composition that inhibits the production of oxidized LDL”, or a “composition that inhibits the progression of atherosclerosis”.

### EFFECT OF THE INVENTION

Based on the atherosclerosis progression inhibiting action possessed by enzymatically modified isoquercitrin, the composition of the present invention, which has enzymatically modified isoquercitrin as an effective component, can exhibit the ability to inhibit the progression of atherosclerosis, the ability to prevent the appearance of atherosclerotic disease, or the ability to ameliorate atherosclerotic disease, in a person at risk for the progression of atherosclerosis, for example, a person with hyperlipidemia or trending in that direction. The composition of the present invention can therefore be effectively utilized as a drug composition (anti-atherogenic agent) whose objective or effect is the prevention or amelioration of atherosclerotic disease. In addition, the composition of the present invention can be effectively utilized as a health food (including, for example, a Food for Specified Health Uses for which a beneficial effect on health can be indicated) whose objective or effect is the prevention or amelioration of atherosclerotic disease.

Furthermore, by inhibiting the progression of atherosclerosis, the composition of the present invention, which has enzymatically modified isoquercitrin as an effective component, is effective for preventing or ameliorating the development of various diseases (atherosclerotic diseases) caused by sclerosis of the arteries (coronary arteries, cerebral arteries, aorta, renal artery, peripheral arteries, and so forth), such as ischemic heart disease (e.g., myocardial infarction, angina pectoris, and so forth), ischemic brain disease (cerebral infarction and so forth) and cerebral hemorrhage, aortic aneurysm and aortic dissection, nephrosclerosis and renal failure arising therefrom, and arteriosclerosis obliterans.

### BEST MODE FOR CARRYING OUT THE INVENTION

The anti-atherogenic composition of the present invention contains enzymatically modified isoquercitrin (referred to below simply as “EMIQ”) as an effective component.

This “enzymatically modified isoquercitrin” is obtained by the action of a glycosyltransferase on isoquercitrin in the presence of a sugar donor and refers to a mixture of isoquercitrin with α-glycosylisoquercitrins having various degrees of glucosylation, as shown by the following formula.

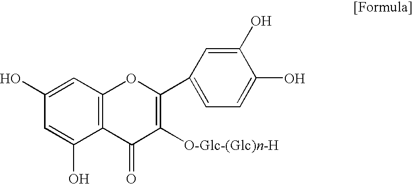

(In the formula, Glc represents a glucose residue and n is 0 or an integer with a value of at least 1.)

Referring to the preceding formula, EMIQ specifically is a mixture of isoquercitrin (the number n of α-1,4-linked glucose residues is 0) with α-glycosylisoquercitrins in which the number n of α-1,4-linked glucose residues is at least 1, generally from 1 to 15, and preferably from 1 to 10.

The EMIQ used by the present invention may be a mixture of different EMIQs having different numbers n of glucose residues bonded therein or may be a single type of EMIQ having a single number n of glucose residues bonded therein.

This EMIQ can be produced by treating isoquercitrin (referred to below simply as IQC) with a glucosyltransferase. While not intended as a limiting description, the EMIQ can generally be produced by glycosylation by effecting the transfer of at least an equimolar amount of glucose residues to IQC using a glucosyltransferase, e.g., glucosidase, transglucosidase, and so forth.

The glucose source used for glycosylation can be a glucose source that enables the transfer of at least one glucose residue molecule therefrom to one molecule of IQC, and can be exemplified by glucose, maltose, amylose, amylopectin, starch, liquefied starch, saccharified starch, cyclodextrin, and so forth. The amount of glucose source used can be exemplified by generally 0.1 to 20 weight parts, preferably 0.5 to 15 weight parts, and more preferably 1 to 10 weight parts, in each case per 1 weight part of the IQC present in the reaction system.

For example, α-amylase (E.C.3.2.1.1), α-glucosidase (E.C.3.2.1.20), and so forth can be used as the glucosidase, while, for example, cyclodextrin glucanotransferase (E.C.2.4.1.19) (abbreviated below as CGTase) and so forth can be used as the transglucosidase.

CGTase is known to be produced by bacteria such as, inter alia, Bacillus species such as Bacillus circulans, Bacillus macerans, Bacillus stearothermophilus, Bacillus megaterium, Bacillus polymyxa, and so forth, and Klebsiella species such as Klebsiella pneumoniae and so forth, and any of these may be used in the present invention without restriction.

These glucosyltransferases are all enzymes that can be acquired commercially, and their commercially available enzyme preparations (for example, product name: Contizyme from Amano Enzyme Inc.) can also be used for the sake of convenience. Purification of these enzymes is not necessarily required, and they can take the form of the crude products. The EMIQ can be produced, for example, by inoculating glucosyltransferase production bacteria into a medium to which IQC has been added, and then allowing them to react by fermentation. It is also possible to produce the EMIQ by immobilizing the glucosyltransferase or the glucosyltransferase production bacteria, and then reacting them with IQC either in a batch or continuous mode. As the glucosyltransferase, it is possible to use a glucosidase or a transglucosidase singly or in a combined manner (simultaneously or sequentially).

The reaction conditions for the glucosyltransferase may be conditions under which the glucosyltransferase is active in a mixed aqueous system comprising IQC, the glucosyltransferase, and a glucose source as described above. The amount of glucosyltransferase used when the glucosyltransferase is CGTase (approximately 100 units of enzyme specific activity where 1 unit is the amount of enzyme that produces 1 mg of β-cyclodextrin per 1 minute from soluble starch) can be suitably selected from the range of 0.001 to 20 weight parts per 1 weight part IQC. Approximately 0.005 to 10 weight parts is preferred and approximately 0.01 to 5 weight parts is more preferred.

There are no particular limitations on the amount of IQC in the reaction system; however, with the goal of carrying out glycosylation in an efficient manner, the IQC is desirably present therein generally at 0.1 to 30 weight %, preferably 0.5 to 20 weight %, and more preferably 1 to 10 weight % with the reaction system being considered as 100 weight %.

The temperature of this reaction system will vary with the type of enzyme used, but temperatures suitably selected from the range of approximately not more than 80° C. can be used. Within this range, approximately 20 to 80° C. and preferably approximately 40 to 75° C. are industrially advantageous. The pH is generally approximately 3 up to approximately 11 and preferably is 4 to 8.

The reaction can be carried out at quiescence or with stirring or shaking. In order to inhibit oxidation during the reaction, the reaction system's headspace may be substituted with an inert gas, for example, nitrogen and so forth, and an antioxidant, for example, ascorbic acid and so forth, may also be added to the reaction system.

Proceeding in this manner, the glucose group is bonded to the glucose residue of IQC to produce the desired EMIQ.

The number of glucose groups bonded to the glucose residue of IQC (the number n in the preceding formula (1)) is not particularly limited, but can as desired be adjusted so as to be in the range generally of 1 to 15 as described above and preferably 1 to 10. In one example of a procedure for carrying out this adjustment, production of the EMIQ is followed by treatment with a single selection, or with the combination of a plurality of selections, from various amylases (for example, α-amylase, β-amylase, glucoamylase, α-glucosidase, maltase, and so forth). Doing this makes it possible to reduce the number of glucose sugar chains in the EMIQ molecule obtained by the above-described method and also makes it possible to obtain EMIQ having any glucose chain length.

The method of isolating and/or purifying the EMIQ from the above-described reaction system is also not particularly restricted. The isolation procedure can be exemplified by isolation using a gel filtration resin employing the usual techniques. The procedure for purifying the EMIQ is not particularly limited, and purification can be carried out using any combination of conventional techniques. Specific examples are the various resin treatment techniques (e.g., adsorption, ion exchange, gel filtration, and so forth), techniques involving treatment with a membrane (e.g., treatment with an ultrafiltration membrane, treatment with a reverse osmosis membrane, treatment with an ion-exchange membrane, zeta potential membrane treatment, and so forth), electrodialysis, salting out, acid precipitation, recrystallization, solvent fractionation techniques, treatment with active carbon, and so forth.

The EMIQ obtained in this manner is readily soluble in water and has as its main component a-glycosylisoquercitrin in which at least equimolar glucose is additionally bonded to the glucose residue of IQC (quercetin 3-O-monoglucoside).

Atherosclerotic diseases denote diseases whose development is attributable to atherosclerosis. More specifically, atherosclerosis disease is a general term for diseases caused by impaired blood circulation to a tissue or an entire organ due to such factors as an artery undergoing sclerosis due to a loss of elasticity, constriction or occlusion of the lumen due to deposition therein, enlargement of all or part of an arterial wall (arterial aneurysm, hypertrophy), and intimal tearing and medial splitting (dissection) or rupture (ischemia). These diseases can be specifically exemplified by cerebral infarction and brain hemorrhage (cerebral arteries), ischemic heart diseases such as myocardial infarction, angina pectoris, and so forth (coronary arteries), aortic aneurysm and aortic dissection (aorta), nephrosclerosis and renal failure arising therefrom (renal artery), and arteriosclerosis obliterans (peripheral arteries).

As shown in the test examples described below, the EMIQ, because it can inhibit the progression of atherosclerosis in warm-blooded animals, including humans, at risk for the progression of atherosclerosis (for example, hyperlipidemia or trending in that direction), can be effectively used for preventing or ameliorating the development of atherosclerotic diseases. With regard to arteriosclerosis, the EMIQ in particular has an excellent capacity to inhibit the progression of atherosclerosis. As a consequence, the EMIQ can be effectively used as a component of an anti-atherogenic composition.

The “anti-atherogenic composition” of the present invention denotes a composition that has the ability to inhibit the progression of atherosclerosis and that can be effectively used to prevent or ameliorate the progression of atherosclerosis and the development of atherosclerotic diseases that originate with atherosclerosis. More preferably, it is a composition that has an anti-atherogenic efficacy and in particular that has the capacity to inhibit or ameliorate the progression of atherosclerosis and that is used for this specific health use. This composition can take the form, for example, of a drug composition, a Food for Special Dietary Use (e.g., medical food for the ill, food for the elderly, and so forth), a Food with Health Claims (e.g., a Food with Nutrient Function Claims, a Food for Specified Health Uses, and so forth), and health foods corresponding thereto.

A Food for Specified Health Uses (including qualified Food for Specified Health Uses, standardized Food for Specified Health Uses, and reduction of disease risk Food for Specified Health Uses) is a food that can make, for example, on its packaging container, a functional claim or a health use indication that has been approved or recognized by the Japanese Ministry of Health, Labor and Welfare. However, a Food for Specified Health Uses is not limited to this, and a Food for Specified Health Uses can make an indication, with respect to an individual who seeks a diet with a specified health objective, to the effect that said health objective can be expected when the food is consumed. The specific health objective in the present invention is anti-atherogenesis and specifically the inhibition or amelioration of the progression of arteriosclerosis (preferably atherosclerosis), and the indication can be exemplified by “inhibits or prevents atherosclerosis”, “improves blood flow (circulation)”, “maintains vascular health”, “for someone with high cholesterol”, or “for someone with high neutral lipids”. A Food for Specified Health Uses, given that these functional claims can be made for such a food, is a very suitable embodiment of the food encompassed by the present invention since this enables a differentiation to be made from ordinary foods that are unable to make functional claims.

To prepare a drug composition, the EMIQ can be made into various orally administratable formulations, e.g., a liquid form (e.g., emulsions, solutions, or drinks such as syrups) in which it is dissolved or dispersed in water, an alcohol (for example, ethanol), or another solvent, or a solid form (e.g., powders, granules, tablets, pills, capsules, chewable forms, and so forth) prepared or formed by known methods.

These formulations may contain, in addition to the aforementioned EMIQ, pharmaceutically acceptable carriers and additives in correspondence to the particular form of administration. Excipients for the preparation of a solid formulation can be exemplified by lactose, sucrose, glucose, corn starch, gelatin, starch, dextrin, calcium phosphate, calcium carbonate, natural and synthetic aluminum silicates, magnesium oxide, dried aluminum hydroxide, magnesium stearate, sodium bicarbonate, dried yeast, and so forth. Excipients for the preparation of a liquid formulation can be exemplified by water, glycerol, propylene glycol, simple syrup, ethanol, ethylene glycol, polyethylene glycol, sorbitol, and so forth. As desired, these formulations can be mixed with the usual additives, e.g., stabilizers such as citric acid, phosphoric acid, malic acid, and their salts; high-intensity sweeteners such as sucralose and acesulfame potassium; sweeteners such as sucrose and fructose; preservatives such as alcohols and glycerol; as well as demulcents, diluents, buffers, flavorants, colorants, and so forth, and can be produced, by the usual methods or other suitable methods, as various orally administratable formulations, such as powders, granules, tablets, capsules, chewable formulations, emulsions, solutions, syrups, and so forth.

The drug form of the subject anti-atherogenic composition is preferably formulated so as to contain from 3 mg to 30 g of the EMIQ per unit of daily administration. This dose can be adjusted as appropriate in response to various factors, e.g., the health status of the individual taking the EMIQ, the mode of administration, the combination with other agents (or food components), and so forth. It is preferably 8 mg to 10 g and more preferably is 16 mg to 2 g.

The EMIQ is also useful as a component of a health food or animal feed used for the purpose of inhibiting the progression of atherosclerosis, preventing the appearance of atherosclerotic diseases, and/or ameliorating atherosclerotic diseases, and is targeted to warm-blooded animals, including humans, at risk for the progression of atherosclerosis (for example, hyperlipidemia or trending in that direction).

Here, health food denotes a food having the goal, inter alia, of maintaining or promoting health in a more active way than ordinary food. By production in a formulated form as described above (powder, granule, tablet, capsule, chewable, emulsion, solution, or syrup) using vehicles and additives acceptable for food applications, the EMIQ in this invention can also be provided as a supplement whose purpose is the inhibition of the progression of atherosclerosis and the prevention or amelioration of atherosclerotic diseases. Moreover, through its addition to ordinary foods (in other words, through its use as a food ingredient), the EMIQ of the present invention can be used to produce health foods (for example, a functional food or a Food for Specified Health Uses) whose objective, function, or effect is the inhibition of the progression of atherosclerosis and the prevention or amelioration of atherosclerotic diseases.

There are no particular limitations on the type of food, and the following can be provided as examples: (1) beverages such as dairy beverages, lactic acid bacteria drinks, fruit juice-containing soft drinks, soft drinks, carbonated beverages, fruit juice beverages, vegetable beverages, vegetable and fruit beverages, powdered beverages, coffee beverages, black tea beverages, green tea beverages, dairy beverages, soy milk beverages, cocoa, jelly-containing beverages, sports drinks, supplement drinks, green juices, and so forth; (2) desserts, e.g., puddings such as custard pudding, milk pudding, soufflé pudding, fruit juice-containing pudding, and so forth, and also jellies, Bavarian cream, and so forth; (3) frozen confections such as ice cream, ice milk, lacto-ice, milk ice cream, fruit juice-containing ice cream and soft-serve ice cream, ice candy, sherbet, ices, and so forth; (4) gums (stick gum, sugar-coated granular gum) such as chewing gum, bubble gum, and so forth; (5) chocolates such as coated chocolate (e.g., chocolate bars, chocolate pieces) as well as flavored chocolate such as strawberry chocolate, blueberry chocolate, melon chocolate, and so forth; (6) candies such as hard candies (bonbons, butterballs, marbles, and so forth), soft candies (caramels, nougats, gummy candies, marshmallows, and so forth), drops, taffy, and so forth; (7) baked/fried confections such as hard biscuits, cookies, okaki rice crackers, senbei rice crackers, and so forth (the preceding (2) to (7) are collectively referred to as confections); (8) seasonings and condiments such as ketchup, sauces, soy sauce, dressings, miso, sugar, salt, mayonnaise, vinegar, black vinegar, and so forth; (9) meat products such as ham, sausage, bacon, frozen hamburger, and so forth; (10) quasi-drugs and dietary supplement foods such as intraoral drugs (e.g., mouth spray and so forth), lozenges, swallowing aids, drinkable preparations, granules, powders, tablets, and so forth; (11) pastes such as marmalade, jam, margarine, butter, flour paste, peanut butter, and so forth; (12) boiled fish-paste products such as fish ham, fish sausage, kamaboko, fish paste sausage, pounded fish cake, tempura, and so forth; (13) retortable products such as retortable curry, retortable soup, retortable stew, and so forth; (14) various noodle dishes such as udon, soba, Chinese noodles, spaghetti, macaroni, dried noodles, somen noodles, instant noodles, and so forth; (15) alcoholic beverages such as wines (e.g., red wine and so forth), liquor, shochu based beverages, carbonated alcoholic beverages, and so forth; (16) processed soy foods such as tofu, fried tofu, and so forth; (17) fermented or cultured foods, such as Japanese pickles, cheese, yogurt, tempeh, natto, and so forth; and (18) pet foods such as dog food, cat food, and so forth.

Considered from the perspective of ongoing consumption, very suitable examples are confections and other solid products, such as breads, candies, tablet confections, and so forth; liquid products such as soft drinks and nutritional drinks; and semisolid products such as jellies. Beverages are more preferred.

The food form of the anti-atherogenic composition is preferably produced so as to contain the EMIQ at 3 mg to 30 g per daily unit of administration. This dose can be adjusted as appropriate in response to various factors, e.g., the health status of the individual taking the EMIQ, the mode of ingestion, the combination with other agents (or food components), and so forth. It is preferably 8 mg to 10 g and more preferably is 16 mg to 2 g.

When in particular the anti-atherogenic composition is produced in beverage form, the EMIQ is preferably blended so as to provide 3 mg to 30 g, preferably 8 mg to 10 g, and more preferably 16 mg to 2 g EMIQ per 500 mL of beverage volume, although these proportions are not to be construed as limiting.

### EXAMPLES

Preparation examples, test examples, and examples are described below in order to more fully elucidate the structure and effects of the present invention. However, the present invention is in no way affected by these examples.

### Preparation Example 1

### Preparation of IQC

250 g flower buds from the Japanese pagoda tree (Saphora japonica), a member of the Fabaceae family, was soaked for 2 hours in 2500 mL hot water (at least 95° C.) and then filtered and the resulting filtrate was recovered and designated as the first extract. The filtered off residue was subjected to extraction by additional soaking in hot water to yield a second extract. The first and second extracts were combined, cooled to 30° C. or below; the precipitated component was filtered off; and the precipitate was washed with water, recrystallized, and dried to yield 22.8 g rutin with a purity of at least 95%.

20 g of this rutin was dispersed in 400 mL water and the pH was adjusted to 4.9 using a pH adjustment agent. To this was added 0.12 g naringinase (Amano Enzyme Inc., product name: Naringinase “Amano”, 3,000 U/g) to start a reaction followed by holding for 24 hours at 72° C. The reaction solution was thereafter cooled to 20° C. and the precipitate produced by cooling was filtered off. The obtained precipitate (solid fraction) was washed with water and dried, yielding 13.4 g isoquercitrin (IQC).

### Preparation Example 2

### Preparation of EMIQ

500 mL water and then 40 g corn starch were added to 10 g of the IQC obtained as described above and a dispersion was prepared. To this was added 15 g cyclodextrin glucanotransferase (CGTase, Amano Enzyme Inc., product name: Contizyme, 600 U/mL) and a reaction was started, followed by holding for 24 hours at pH 7.25 and 60° C. The resulting reaction solution was then cooled and loaded on a column (Φ3.0×40 cm) of Diaion HP-20 (Mitsubishi Chemical Corporation), which was thereafter washed with 1000 mL water. 600 mL 50% aqueous ethanol was then fed to the column, and the resulting eluate was concentrated under reduced pressure and freeze-dried to yield 12.8 g enzymatically modified isoquercitrin (EMIQ).

The obtained EMIQ was submitted to HPLC analysis under the conditions given below, and the molar proportions (%) of the various α-glycosylisoquercitrins present in the EMIQ were calculated.

### <HPLC Conditions>

column: Inertsil ODS-2Φ4.6×250 mm (GL Sciences Inc.)
eluent: water/acetonitrile/TFA=850/15/2
detection: absorbance measured at a wavelength of 351 nm
flow rate: 0.8 mL/min.

The molar composition proportions (%) in the EMIQ are shown below. These molar composition proportions are the composition proportions calculated using the sum of the 8 components from IQC to IQC+Glc7 (7 glucoses bonded by α-1,4-linkages to IQC) as 100%. In addition to these components, the EMIQ contained trace amounts of products in which 8 or more glucoses were bonded to IQC (IQC+Glc8 or higher).

TABLE 1

| | | IQC + | IQC + | IQC + | IQC + | IQC + | IQC + | IQC + | |
|---|---|---|---|---|---|---|---|---|---|
| component | IQC | Glc1 | Glc2 | Glc3 | Glc4 | Glc5 | Glc6 | Glc7 | total |
| molar | 31.6 | 23.4 | 19.9 | 10.1 | 7.0 | 4.4 | 2.4 | 1.2 | 100.0 |
| composition | | | | | | | | | |
| proportion | | | | | | | | | |
| (%) | | | | | | | | | |

IQC: isoquercitrin
Number in Glc1 to Glc7: number of glucoses added to IQC

### Preparation Example 3

### Production of Quercetin (QC)

500 mL of 2 N sulfuric acid aqueous solution was added to 10 g of the IQC obtained in Preparation Example 1, followed by hydrolysis for 2 hours on a boiling water bath. The quercetin was precipitated by cooling the resulting reaction solution to 30° C. or below; filtration then yielded crude quercetin. The obtained crude quercetin was washed with water, recrystallized, and dried to recover 5 g quercetin (QC) with a purity of at least 95%.

### Test Example 1

### Influence of EMIQ On Atherosclerosis (1)

The EMIQ prepared in Preparation Example 2 was orally administered for 14 weeks to hyperlipidemic ApoE knock out mice (ApoE-KO mice) that had been rendered deficient in the apolipoprotein E gene (ApoE) by genetic manipulation, in order to elucidate the atherosclerosis progression inhibiting effect of this substance. The atherosclerosis progression inhibiting effect was evaluated based on the atherosclerotic lesion area in the thoracic-abdominal aorta of the ApoE-KO mice and based on the plaque area at the aortic valve of the ApoE-KO mice.

### 1. Test Animals And Housing Conditions

For the test animals, B6.129P2-Apoe<tm1Unc>mice (ApoE-KO mice, age: 6 weeks after birth, male) were purchased from Jackson Laboratory (USA); testing was begun after a 4-day period of housing for environmental acclimation. Three or four test animals (ApoE-KO mice) were housed per cage in polycarbonate cages (W 182×D 260×H 128 mm) and were kept in a housing facility that had an illumination interval of 12 hours (7:00 to 19:00) and that was controlled to a room temperature of 21 to 26° C. and a humidity of 40 to 70% by the half-return regime with a microorganism-excluding HEPA filtered air supply. During the acclimation period, Mouse·Rat MF Solid Feed (Oriental Yeast Co., Ltd., Tokyo) was made available. After the start of the test, a feed based on a mouse atherogenesis-inducing feed (20% milk casein, 46.54% granulated sugar, 1% corn oil, 20% cocoa butter, 4.82% KC Flock, 0.15% cholesterol, 0.06% cholic acid, 1% vitamin mix (AIN-76), 5.0% mineral mix (AIN-76), 1.0% choline chloride, DL-α-tocopherol, and 0.3% DL-methionine; from Nippon Formula Feed Mfg. Co., Ltd., Tokyo) was made available continuously. Well water (sterilized by chlorination and ultraviolet irradiation) was made available to the mice ad libitum from a water bottle. The body weight range at the start of the test was 20 to 25 g.

At the start of the test, the test animals (ApoE-KO mice) were assigned to two groups, a control group (EMIQ non-administration group) and an EMIQ group (EMIQ administration group), taking in account body weight in a completely randomized design using a group assignment system (Statlight #11 Group Assignment, Yukms Co., Ltd., Tokyo).

### 2. Test Methods

### (1) Sample Administered And Its Method of Administration

The EMIQ used was the EMIQ prepared by the method described in the preceding Preparation Example 2. The EMIQ was mixed into the mouse atherogenesis-inducing feed so as to give a concentration of 330 μmol/kg (0.026 w/w %) and was thereby orally administered (feed-admixed administration, feed available continuously) over 14 weeks to the EMIQ group of test animals. The mouse atherogenesis-inducing feed alone, lacking the EMIQ, was orally administered (feed available continuously) to the control group over a 14-week period.

### (2) Inspection of General Condition And Body Weight Measurement

After the start of the test, the body weight was measured and the general condition was inspected once each day for the test animals (10 animals in each group) in both groups (control group, EMIQ group).

### (3) Necropsy

In the 14th week after the start of the test, the test animals in each group were euthanized under sodium pentobarbital anesthesia (intraperitoneal administration) by exsanguination and blood collection with an untreated syringe. The aorta was irrigated and fixed with 2% neutral buffered formalin solution, and the thoracic-abdominal aorta was extirpated after longitudinal sectioning precisely in the middle while still attached in the body and opening. While in an opened state, the extirpated thoracic-abdominal aorta was immersed and fixed in 4% paraformaldehyde solution. The thoracic-abdominal aorta was then stained with Oil red-O. In addition, the aortic arch and aortic valve section was stained with HE and Elastica Van Gieson (EVG) by the usual methods.

### (4) Image Analyses

The aortic valve plaque area was analyzed using a VM-30 micrometric tablet measurement unit (Olympus Corporation, Tokyo), and the atherosclerotic lesion area in the thoracic-abdominal aorta was analyzed using an IPAP image processor for analytical pathology (Sumika Technoservice Corporation, Osaka). Specifically, the intimal thickening (%) at the aortic valve and the atherosclerotic lesion area rate (%) in the thoracic-abdominal aorta were calculated using the following formulas.

aortic valve plaque area rate (%)=intimal area÷total area of the aortic valve circumferential transverse section×100 (i)

thoracic-abdominal aortic atherosclerotic lesion area rate (%)=area of dark red staining÷total blood vessel area×100 (ii)

The numerical values obtained in the experiments were used to calculate an average value and standard deviation for each test group (control group, EMIQ group). With regard to the test of significant difference, the equality of variance was tested by the F-test, and the test of significant difference was carried out by Student's t-test in the case of equal variance and was carried out by Aspin-Welch's t-test in the case of unequal variance. With regard to the significance level, significance was set at 20% for the F-test and was set at less than 5% for the others and is shown classified into less than 5% (p<0.05) and less than 1% (p<0.01).

### 3. Test Results

### (1) General Condition And Body Weight

Throughout the test period, the body weight underwent a smooth increase in both groups (control group, EMIQ group) and no difference was observed between the groups. In addition, no noteworthy changes in general condition were seen in either group.

### (2) Results of Image Analysis (Aortic Valve Plaque Area And Atherosclerotic Lesion Area Rate In the Thoracic-Abdominal Aorta)

The aortic valve plaque area rate (%) and the atherosclerotic lesion area rate (%) in the thoracic-abdominal aorta are shown in Table 2 for both groups (control group and EMIQ group). The results for the atherosclerotic lesion area rate (%) at the thoracic-abdominal aorta are shown in FIG. 1 for the individual test animals in the respective groups (control group (n=10), EMIQ (n=9)).

TABLE 2

| | | atherosclerotic lesion |
|---|---|---|
| | aortic valve plaque | area rate (%) at the |
| | area rate (%) | thoracic-abdominal aorta |
| control group | 4.45 ± 3.02 | 9.49 ± 4.11 |
| EMIQ group | 2.80 ± 2.39 | 5.61 ± 2.48 |

As may be understood from the table, the aortic valve plaque area rate (%) was significantly less (p<0.05, Student's t-test) in the EMIQ group (n=68) at 2.80±2.39% (average±standard deviation) than in the control group (n=36) with its 4.45±3.02%. In addition, the atherosclerotic lesion area rate (%) at the thoracic-abdominal aorta was also significantly less (p<0.05, Student's t-test) in the EMIQ group (n=9) at 5.61±2.48% (average±standard deviation) than the 9.49±4.11% of the control group (n=10).

These results demonstrated that EMIQ has an atherosclerosis progression inhibiting effect. From a dietary perspective, it is thought that, given the absence of negative effects on the body, EMIQ is effective for preventing atherosclerosis through the its continual ingestion.

### Test Example 2

### Influence of EMIQ On Atherosclerosis (2)

The EMIQ prepared in Preparation Example 2 and the QC prepared in Preparation Example 3 were orally administered for 14 weeks to ApoE-KO mice in order to elucidate the atherosclerosis progression inhibiting effect of these substances. The atherosclerosis progression inhibiting effect was evaluated based on the atherosclerotic lesion area rate (%) at the thoracic-abdominal aorta of the ApoE-KO mice. The test animals and their housing conditions and the test method (measurement of the atherosclerotic lesion area rate (%) at the thoracic-abdominal aorta) were as described in Test Example 1.

Specifically, before the test the test animals (ApoE-KO mice) were housed for 11 days for environmental acclimation and were then assigned, taking into consideration the body weight and total serum cholesterol (T-cho value), into the following four groups (8 animals in each group) in a completely randomized design using a group assignment system (Statlight #11 Group Assignment, Yukms Co., Ltd., Tokyo): the control group (a group not receiving either substance, G1), a group receiving quercetin admixed in the feed at 33 μmol/kg (low-dose QC group, G2), a group receiving EMIQ admixed in the feed at 33 μmol/kg (low-dose EMIQ group, G3), and a group receiving EMIQ admixed in the feed at 330 μmol/kg (high-dose EMIQ group, G4). For these 4 groups, the particular test substance was administered admixed in the mouse atherogenesis-inducing food (feed-admixed administration, feed available continuously) in the case of the groups receiving a test substance, while only the mouse atherogenesis-inducing food was provided (feed available continuously) in the control group (group not receiving either substance). In specific terms, the mouse atherogenesis-inducing food was continuously provided for 14 weeks to the control group; the mouse atherogenesis-inducing food supplemented with QC at a concentration of 33 μmol/kg was continuously provided for 14 weeks to the low-dose QC group; the mouse atherogenesis-inducing food supplemented with EMIQ at a concentration of 33 μmol/kg was continuously provided for 14 weeks to the low-dose EMIQ group; and the mouse atherogenesis-inducing food supplemented with EMIQ at a concentration of 330 μmol/kg was continuously provided for 14 weeks to the high-dose EMIQ group. This was followed by necropsy of all the test animals and determination of the thoracic-abdominal aortic atherosclerotic lesion area rate (%) according to the methodology of Test Example 1.

The results are shown in FIG. 2. The value (%) shown here is the average value of the thoracic-abdominal aortic atherosclerotic lesion area rate (%) for the mice (8 animals) in the particular group. As shown in FIG. 2, there was no difference in the thoracic-abdominal aortic atherosclerotic lesion area rate (%) between the control group (G1) and the mice in the low-dose QC group (G2), while in contrast the thoracic-abdominal aortic atherosclerotic lesion area rate (%) in the mice in the low-dose EMIQ group (G3) was found to be substantially lower than that in the control group (G1). In addition, the thoracic-abdominal aortic atherosclerotic lesion area rate (%) in the mice in the high-dose EMIQ group (G4) was almost unchanged from that of the low-dose EMIQ group (G3). Based on these results, it may be understood that low-dose EMIQ is significantly different from QC at an equimolar concentration and has the ability to inhibit atherogenesis in the aorta, which is the initial pathological change in atherosclerosis, that is, low-dose EMIQ has the ability to inhibit the progression of atherosclerosis.

### Test Example 3

In order to investigate the mechanism of the atherosclerosis progression inhibiting effect exhibited by EMIQ in Test Examples 1 and 2, the aortic valve plaque lesion was immunostained by various staining methods (4HNE staining, Mac3 staining, Masson's trichrome, αSM staining) and the stained area rates (%) were determined.

Specifically, proceeding as in Test Example 2, the test animals (ApoE-KO mice) were preliminarily housed for 11 days for environmental acclimation and were then assigned to the following three groups (8 animals per group): the control group (non-administration group), a group receiving quercetin admixed in the feed at 330 μmol/kg (QC group), and a group receiving EMIQ admixed in the feed at 330 μmol/kg (EMIQ group).

These groups were provided, respectively, with the following on a continuous basis for 14 weeks (feed available continuously): the mouse atherogenesis-inducing feed, the mouse atherogenesis-inducing feed to which QC had been added, and the mouse atherogenesis-inducing feed to which EMIQ had been added. Then, proceeding as in (3) in Test Example 1, euthanasia was carried out and transverse-sectioned aortic valve specimens were prepared and immunostaining (hematoxylin staining, 4HNE staining, Mac3 staining, αSM staining) was carried out by the usual methods; Masson's trichrome staining was carried out as a specialty stain. The stained area at the aortic plaque for these specimens was measured using fluorescent image analysis system software (Lumina Vision ver. 2.20, Mitani Corporation). The stained area rate (%) was calculated using the following equation.

stained area/total plaque area×100=stained area rate (%)

4HNE staining, Mac3 staining, αSM staining, and Masson's trichrome staining were performed as follows.

### (1) 4HNE Staining

The transverse-sectioned aortic valve specimen was reacted with primary antibody (anti-4HNE monoclonal antibody: Nikken SEIL Corporation, Japan Institute for the Control of Aging) against 4-hydroxy-2-nonenal (4HNE), a marker of lipid oxidation, and the 4HNE was then specifically stained using a VECTASTAIN DAB substrate kit (Funakoshi Corporation). This method enables the localization and quantification of oxidative stress in tissue.

### (2) Mac3 Staining

Macrophages were specifically stained by reacting the transverse-sectioned aortic valve specimen with a primary antibody (anti-Mac-3 monoclonal antibody: BD Bioscience) against Mac-3, which is a macrophage-specific surface glycoprotein. This method enables the localization and quantification of macrophages in tissue.

(3) αSM staining

Differentiated smooth muscle cells were specifically stained by reacting the transverse-sectioned aortic valve specimen with primary antibody (anti-α-smooth muscle actin monoclonal antibody: SIGMA) against αSM actin, which is highly expressed in differentiated smooth muscle cells and myofibroblast-type fibroblasts. This method enables the localization and quantification of differentiated vascular smooth muscle in tissue. Differentiated smooth muscle cells are present in the smooth muscle cells of stable vascular walls, and when the vessel wall is subjected to stress they undergo a phenotypic transformation to undifferentiated smooth muscle cells in which there is little αSM actin expression; it is reported that this is connected to the proliferative pathological changes of the atherosclerotic lesion and to unstable plaque.

### (4) Masson's Trichrome Stain

The transverse-sectioned aortic valve specimen was stained according to a reported method (Handbook of Tissue Staining for Pathology, Igaku-Shoin Ltd.) using aniline blue as the staining solution. This stain enables the localization and quantification of collagen fibers by staining the collagen fibers in tissue blue with the aniline blue.

The stained area ratio (%) obtained with each stain is shown in FIG. 3. The values (%) given here are average values for the mice in each group (8 animals). The accumulation of the lipid oxidation marker 4HNE and macrophage infiltration at the plaque lesion site at the aortic valve were significantly inhibited in the EMIQ group (4HNE: p=0.022, Mac3: p=0.006). It can be inferred from this that, in the process in the blood vessels of the experimental atherosclerotic mouse model wherein the pathological changes progress via infiltration of macrophages into the atherosclerotic lesion site and incorporation of oxidized LDL, EMIQ can exhibit an anti-atherogenic activity by a mechanism that acts to inhibit these phenomena. In addition, the staining ratio for αSM actin, which is abundant in differentiated vascular smooth muscle cells, was significantly higher (p=0.035) in the EMIQ group than in the control group. The staining rate for collagen fibers in the case of the Masson's trichrome staining was also significantly higher (p=0.006) in the EMIQ group than in the control group. The progression to an easily ruptured unstable plaque reportedly occurs at the site of an unstable atherosclerotic lesion via a reduction in stable differentiated vascular smooth muscle cells, a reduction in the production of collagen fibers, and a reduction in the extracellular matrix brought about by Matrix metalloproteinase (MMP) secreted by, for example, foam cells. The results given here suggest that EMIQ has a protective action against the reduction in vascular smooth muscle and the reduction in collagen fiber that are connected to plaque destabilization.

### Test Example 4

### Ability To Inhibit the Production of Oxidized Low-Density Lipoprotein (LDL: Low-Density Lipoprotein)

The inhibitory effect on the production of oxidized low-density lipoprotein (oxidized LDL) was investigated using the EMIQ produced in Preparation Example 2. For comparison, the inhibitory effect on the production of oxidized LDL was similarly investigated using the IQC produced in Preparation Example 1.

### 1. Test Method

Test solutions (control, blank, EMIQ-containing solution, IQC-containing solution) were prepared according to the recipes described in Table 3 below and each solution was incubated for 4 hours at 37° C. The test principle (blank) is that a lipid oxidation chain reaction is induced when the radical produced by the conversion of hypoxanthine to xanthine by xanthine oxidase is transferred into the LDL interior via ADP-Fe³⁺ chelate. The low-density lipoprotein (LDL) (human low-density lipoprotein)(Harbor Bio-Products) was used after removal of the EDTA present in this reagent by preliminarily dialysis in 10 mM HEPES-145 mM KCl (pH 7.4). The ADP·Fe³⁺ chelate was prepared by mixing 10 mm adenosine 5′-diphosphate disodium salt (Wako Pure Chemical Industries, Ltd.), 1 mM FeCl₃ (Kishida Chemical Co., Ltd.), and 10 mM HEPES-145 mM KCl (pH 7.4) and incubating for 90 minutes at room temperature. For the EMIQ solution, the test was carried out using an addition equimolar (200 μM) with the IQC concentration in the IQC solution.

TABLE 3

| | control | Blank | | EMIQ solution | | IQC solution | |
|---|---|---|---|---|---|---|---|
| low-density | 100 μg | 100 | μg | 100 | μg | 100 | μg |
| lipoprotein (LDL) | | | | | | | |
| xanthine oxidase | — | 10 | mU | 10 | mU | 10 | mU |
| hypoxanthine | — | 1 | mM | 1 | mM | 1 | mM |
| ADP•Fe³⁺ chelate | — | 1 | mM | 1 | mM | 1 | mM |

| | | | | | |
|---|---|---|---|---|---|
| EMIQ | — | — | 200 | μM | — |

| | | | | | |
|---|---|---|---|---|---|
| IQC | — | — | — | 200 | μM |

After incubation for 4 hours, the absorbance at a wavelength of 532 nm was measured and used as the amount of TBARS (thiobarbituric acid reactive substance) as an index of the oxidized LDL produced in the test solution. The TBARS value for each test solution (blank, EMIQ solution, IQC solution) was corrected by the TBARS value of the control. The TBARS production rate (%) of the EMIQ solution and the IQC solution was calculated by converting the TBARS value for the EMIQ solution and the IQC solution using the TBARS value for the blank as 100%. The results are shown in FIG. 4. These results are the average value of triplicate tests for each test solution.

As may be understood from FIG. 4, the production of oxidized LDL was significantly inhibited (p<0.05, Student's t-test) by the EMIQ solution in comparison to the IQC solution.

The atherosclerotic lesion is known to be produced by the induction of foam cell formation when oxidized LDL produced accompanying lipid peroxidation is taken up by macrophages. As may be understood from the results given above, EMIQ, because it has an excellent capacity to inhibit the production of oxidized LDL, can be effectively used to inhibit the production of the initial pathological change at the atherosclerotic lesion.

### Example 1

### Food Composition (1)

A beverage incorporating 20% grape juice was produced using the EMIQ prepared in Preparation Example 2 as an effective component having an anti-atherogenic activity (atherosclerosis progression inhibiting effect). Specifically, the ingredients according to the following recipe were mixed, filtered, filled into 250 mL bottles, and sterilized for 10 minutes at 80° C. to produce a beverage incorporating 20% grape juice.

| | |
|---|---|
| sucralose | 0.0136 (kg) |
| clear grape juice, 5X concentrate | 4.0 |
| citric acid (crystalline) | 0.25 |
| enzymatically modified isoquercitrin | 0.1 |
| grape flavor | 0.1 |
| fruit flavor | 0.1 |
| water | suitable quantity |
| total | 100.0 kg |

### Example 2

### Food Composition (2)

A sugar-free candy was produced using the EMIQ prepared in Preparation Example 2 as an effective component having an anti-atherogenic activity (atherosclerosis progression inhibiting effect). Specifically, the ingredients according to the following recipe were mixed, dissolved with heating, and molded to produce a sugar-free candy.

| | |
|---|---|
| hydrogenated palatinose | 60.00 (kg) |
| hydrogenated starch syrup | 59.33 |
| sucralose | 0.03 |
| citric acid (anhydrous) | 1.50 |
| trisodium citrate | 0.07 |
| enzymatically modified isoquercitrin | 5.00 |
| grape flavor | 0.20 |
| water | 30.00 |
| cook down to a total quantity of | 100.00 kg |

### Example 3

### Food Composition (3)

A drink jelly was produced using the EMIQ prepared in Preparation Example 2 as an effective component having an anti-atherogenic activity (atherosclerosis progression inhibiting effect). Specifically, the ingredients according to the following recipe were mixed, dissolved with heating, filled into containers, and sterilized for 30 minutes at 85° C. to produced a drink jelly.

| | |
|---|---|
| sugar | 5.00 (kg) |
| high-fructose corn syrup | 15.00 |
| white grape juice, 5X concentrate | 4.00 |
| gelling agent (thickening polysaccharide) | 0.20 |
| gelling agent (gellan gum) | 0.15 |
| trisodium citrate | 0.10 |
| calcium lactate | 0.10 |
| citric acid (anhydrous) | 0.18 |
| enzymatically modified isoquercitrin | 0.18 |
| grape flavor | 0.30 |
| water | suitable quantity |
| total | 100.00 kg |

### Example 4

### Food Composition (4)

Using the EMIQ produced in Preparation Example 2 as an effective component having an anti-atherogenic activity (atherosclerosis progression inhibiting effect), the ingredients according to the following recipe were mixed and tablets were produced using a tableting press.

| | |
|---|---|
| sorbitol | 79.75 (kg) |
| sucrose fatty acid ester | 0.15 |
| flavorant | 0.10 |
| enzymatically modified isoquercitrin | 10.00 |
| total | 100.00 kg |

### BRIEF DESCRIPTION OF THE DRAWINGS

FIG. 1 shows the results measured in Test Example 1 for the atherosclerotic lesion area rate (%) in the thoracic-abdominal aorta for the individual test animals (ApoE-KO mice) in each group (control group (n=10), EMIQ group (n=9)).

FIG. 2 shows the results measured in Test Example 2 for the atherosclerotic lesion area rate (%) in the thoracic-abdominal aorta for the test animals (ApoE-KO mice) in the individual groups (nonadministration group G1, low-dose QC group G2, low-dose EMIQ group G3, high-dose EMIQ group G4) (n=8 in each group).

FIG. 3 shows the results measured in Test Example 3 for the immunostained area rate (%) obtained by immunostaining (4HNE staining, Mac3 staining, Masson's trichrome staining, αSM staining) the aorta of the test animals (ApoE-KO mice) for the individual groups (non-administration group (NON), QC group (QC), and EMIQ group (EMIQ) (n=8 in each group)).

FIG. 4 shows the results from Test Example 4 for the calculation of the TBARS production rate (%), as an index of oxidized LDL, for the solution containing IQC (200 μM)) and for the solution containing EMIQ (200 μM)).

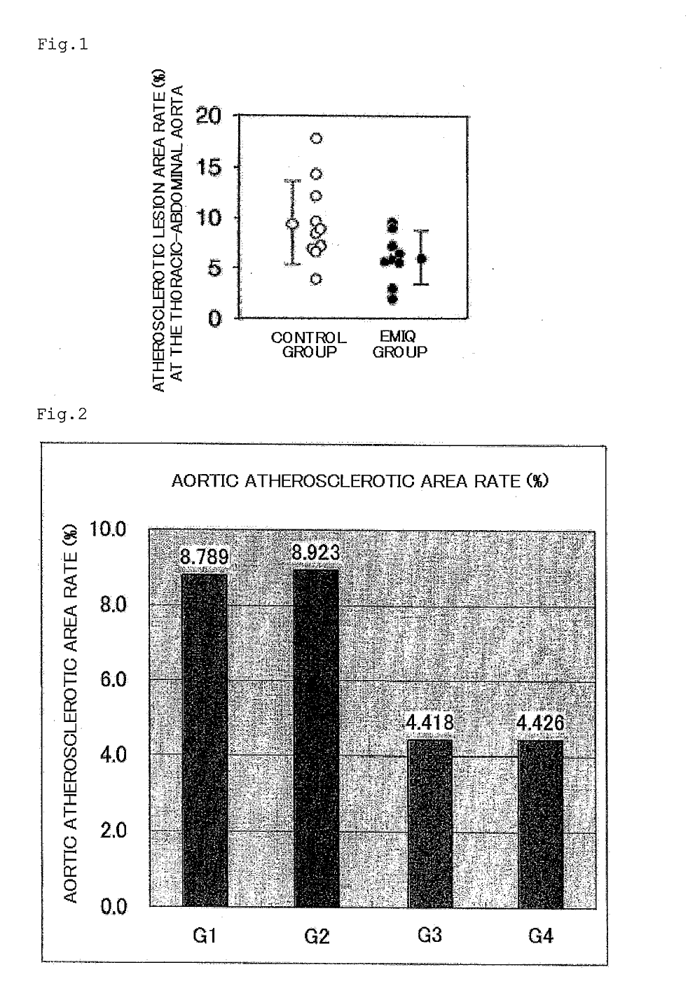

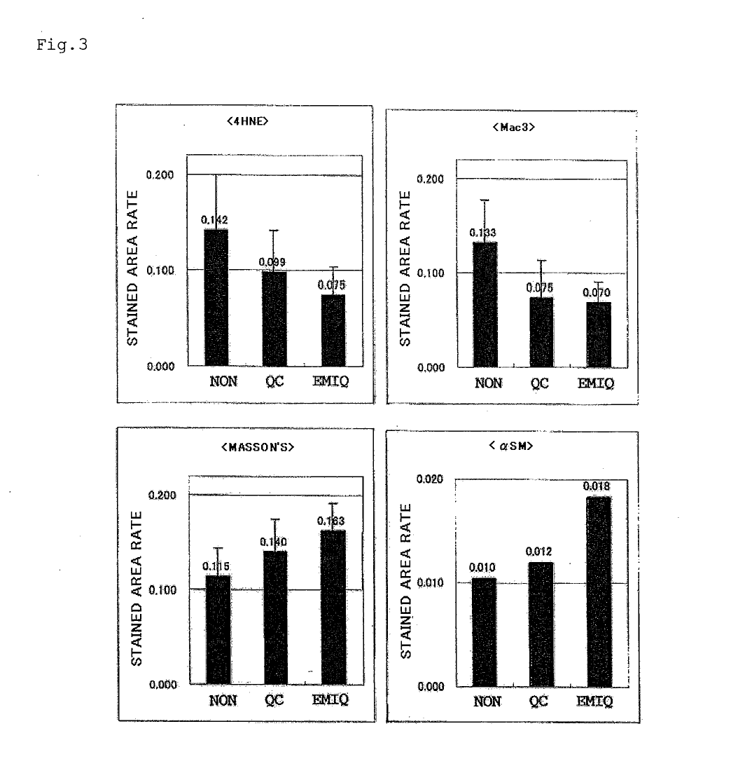

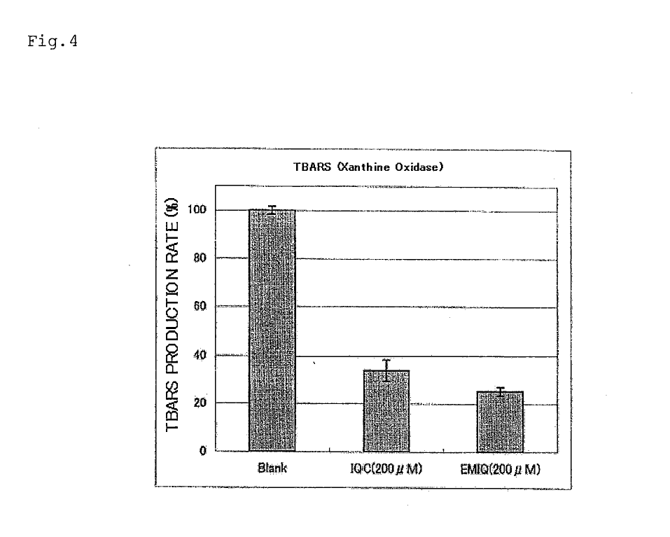

1. A drug composition for inhibiting plaque destabilization comprising an enzymatically modified isoquercitrin as an effective ingredient.

2. The drug composition for inhibiting plaque destabilization according to claim 1, which contains the enzymatically modified isoquercitrin in an amount effective for inhibiting plaque destabilization.

3. The drug composition for inhibiting plaque destabilization according to claim 1, which is in an orally administratable form.

4. The drug composition for inhibiting plaque destabilization according to claim 1, which contains the enzymatically modified isoquercitrin of 3 mg to 30 g per unit of daily administration.

5. The drug composition for inhibiting plaque destabilization according to claim 1, which is formulated so as to contain 3 mg to 30 g of the enzymatically modified isoquercitrin per unit of daily administration.

6. (canceled)

7. (canceled)

8. (canceled)

9. (canceled)

10. A method of inhibiting plaque destabilization in a subject at a risk for progression of atherosclerosis, comprising:
administering to said subject an enzymatically modified isoquercitrin in an amount effective for inhibiting plaque destabilization.

11. A use of an enzymatically modified isoquercitrin for preparation of a composition for inhibiting plaque destabilization.
